(19) United States
(12) Patent Application Publication
(10) Pub. No.: US 2019/0029563 A1
(43) Pub. Date: Jan. 31, 2019

(54) METHODS AND APPARATUS FOR DETECTING BREATHING PATTERNS

(71) Applicant: Intel Corporation, Santa Clara, CA (US)

(72) Inventors: Jelle Sels, Sint-Joris-Weert (BE); Julien Chevrier, Dublin (IE); Srikanth Vasuki, Dublin (IE); William Rafferty, Dublin (IE); Diarmaid O'Cualain, Dublin (IE)

(21) Appl. No.: 15/660,281

(22) Filed: Jul. 26, 2017

(51) Int. Cl. A61B 5/08; A61B 5/00; A61B 7/00

(52) U.S. Cl. CPC A61B 5/0816; A61B 5/6803; A61B 7/003; A61B 5/7203

(57) ABSTRACT

Methods and apparatus for detecting breathing patterns are disclosed herein. An example wearable device includes a frame to be worn by a user in an environment. The example wearable device includes a first microphone carried by the frame. The first microphone is to collect breathing sound data from the user. An example wearable device includes a second microphone carried by the frame. The second microphone is to collect noise data from the environment. The example wearable device includes at least one processor to modify the breathing sound data based on the environmental noise data to generate modified breathing sound data and identify a breathing pattern based on the modified breathing sound data.

### FIELD OF THE DISCLOSURE

This disclosure relates generally to monitoring breathing activity in subjects, and, more particularly, to methods and apparatus for detecting breathing patterns.

### BACKGROUND

Breathing activity in a subject includes inhalation and exhalation of air. Breathing pattern characteristics can include, for example, the rate of inhalation and exhalation, the depth of breath or tidal volume (e.g., a volume of air moving in and out of the subject's lungs with each breath), etc. Breathing patterns may change due to subject activity and/or subject health conditions. Abnormal breathing patterns include hyperventilation (e.g., increased rate and/or depth of breathing), hypoventilation (e.g., reduced rate and/or depth of breathing), and hyperpnoea (e.g., increased depth of breathing).

### BRIEF DESCRIPTION OF THE DRAWINGS

FIG. 1 illustrates an example system constructed in accordance with the teachings disclosed herein and including a wearable device for collecting breathing sound data and a processor for detecting breathing patterns.

FIG. 2 is a block diagram of an example implementation of the breathing pattern detector of FIG. 1.

FIG. 3 is a block diagram of an example implementation of the breathing pattern analyzer of FIG. 1.

FIG. 4 is a flowchart representative of example machine readable instructions that may be executed to implement the example breathing pattern detector of FIG. 2.

FIG. 5 is a flowchart representative of example machine readable instructions that may be executed to implement the example breathing pattern analyzer of FIG. 3.

FIG. 6 illustrates a first example processor platform that may execute one or more of the example instructions of FIG. 4 to implement the example breathing pattern detector of FIG. 2.

FIG. 7 illustrates a second example processor platform that may execute one or more of the example instructions of FIG. 5 to implement the example breathing pattern analyzer of FIG. 3.

The figures are not to scale. Wherever possible, the same reference numbers will be used throughout the drawing(s) and accompanying written description to refer to the same or like parts.

### DETAILED DESCRIPTION

Monitoring a subject's breathing patterns includes obtaining data indicative of inhalations and exhalations by the subject. Breathing pattern characteristics can change with respect to breathing rate, depth of breath or tidal volume, respective durations of inhalations and exhalations, etc. Changes in breathing patterns can result from activities performed by the subject such as exercise. In some examples, breathing pattern data can be used to evaluate a subject's activities and/or health, including stress levels and/or other physiological conditions.

In some examples, an acoustic sensor (e.g., a microphone) is used to record breathing sounds generated as the subject inhales and exhales. However, placing an acoustic sensor under the subject's nose or near the subject's mouth to record breathing sounds can be uncomfortable for the subject and/or may require the subject to be stationary during data collection periods. Conversely, placing the acoustic sensor away from the subject's body may hinder the ability of the sensor to accurately capture breathing sounds. Further, such sensors may not account for ambient sounds from the environment that may be captured by the acoustic sensor and that could interfere with the analysis of the breathing data.

Examples disclosed herein provide for recording of breathing sounds via a first microphone coupled to a head-mounted device (HMD), such as eyeglasses. In some examples, when a user wears the HMD, the first microphone is disposed proximate to the user's nose. The first microphone records audible breathing sounds as the user inhales and exhales. Example HMDs disclosed herein enable breathing data to be gathered while the user is performing one or more activities, such as exercising, relaxing, etc. while reducing (e.g., minimizing) user discomfort.

Example HMDs disclosed herein include a second microphone to record ambient sounds from an environment in which a user wearing the HMD is located while the first microphone records the breathing sound data. Example HMDs disclosed herein include a first processor (e.g., a digital signal processor that is carried by the HMD) to modify (e.g., filter) the breathing sound data generated by the first microphone to remove noise from the breathing sound data (e.g., environmental sounds that may have been captured by the first microphone in addition to the breathing sounds). In some examples, the processor removes the noise by deducting the environmental noise signal data generated by the second microphone from the breathing sound signal data generated by the first microphone. In examples disclosed herein, the processor determines a breathing pattern for the user based on the resulting signal data. Thus, in examples disclosed herein, the breathing pattern is determined based on breathing data that has been filtered to remove or substantially reduce environmental noise data that could interfere with the analysis of the breathing data.

Some example HMDs disclosed herein include a second processor (e.g., a microcontroller) to store the breathing pattern data determined by the processor (e.g., the digital signal processor). In some examples, the second processor analyzes the breathing pattern to determine, for example, breathing efficiency and/or to generate user alerts or notifications. In some examples, the second processor transmits (e.g., via Wi-Fi or Bluetooth connections) the breathing pattern data and/or the results of the analysis to a user device that is different than the wearable device that collects the data (e.g., a smartphone and/or other wearable such as a watch or the like) for further processing and/or presentation (e.g., display) of the results to the user. Examples disclosed herein enable detection and analysis of breathing data collected via the microphone-enabled HMD to provide the user with notifications and/or alerts about his or her breathing performance. In some examples, the breathing data is processed in substantially real-time to provide the user with notifications during user activities via the HMD and/or another user device (e.g., a smartphone, a watch). In some examples, the alert(s) include warnings about potential health conditions detected based on the breathing data, such as an asthma attack. In some examples, the notifications can indicate changes in efficiency breathing and/or provide other breathing metrics that may be monitored as part of a health fitness program.

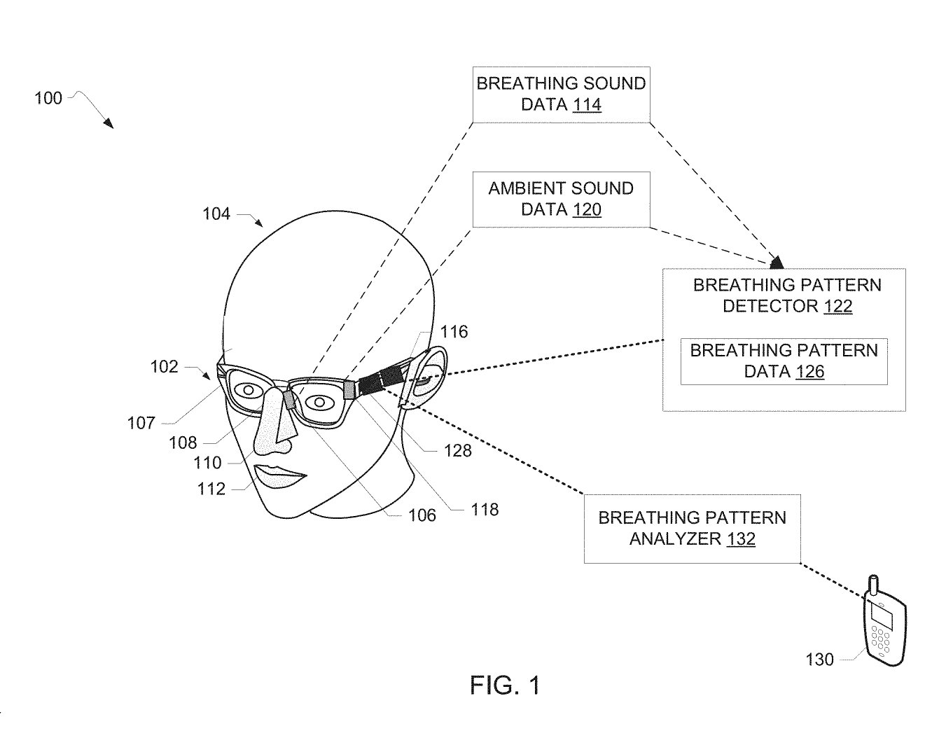

FIG. 1 illustrates an example system constructed in accordance with the teaching of this disclosure for detecting breathing pattern(s) in a subject or user (the terms “user” and “subject” are used interchangeable herein and both refer to a biological creature such as a human being). The example system 100 includes a head-mounted device (HMD) 102 to be worn by a user 104. In the example of FIG. 1, the HMD device 102 includes eyeglasses worn by the user 104. However, the HMD device 102 can include other wearables, such as a mask, ear muffs, goggles, etc.

The HMD device 102 of FIG. 1 includes a first microphone 106 coupled (e.g., mounted) to the HMD 102. In the example of FIG. 1, the first microphone 106 is coupled to a frame 107 of the HMD 102 such that when the user 104 wears the HMD 102, the first microphone 106 is disposed proximate to a bridge 108 of a nose 110 of the user 104. For example, as illustrated in FIG. 1, the first microphone 106 can be coupled to the frame 107 proximate to a nose bridge of the HMD 102 (e.g., the eyeglasses). In other examples, the first microphone 106 is coupled to the HMD 102 at other locations, other components of the HMD 102 (e.g., nose pads) and/or is disposed at other locations relative to the user's face when the HMD 102 is worn by the user 104 (e.g., proximate to the user's dorsum nasi).

In the example of FIG. 1, the first microphone 106 is a high sensitivity microphone capable of detecting quiet sounds associated with breathing and/or lulls in breathing as well as louder sounds from the environment and/or sounds that are generated at a close range to the first microphone 106, such as the user's voice. For example, the first microphone 106 can collect signal data between 120 dB (e.g., corresponding to a sound pressure level for a propeller aircraft) and 33 dB (e.g., corresponding to a sound pressure level for a quiet ambient environment). In some examples, the first microphone 106 is a digital microphone that provides a digital signal output.

The example first microphone 106 detects audible breathing sounds generated by the user 104 during inhalation and exhalation and collects (e.g., records) the breathing sounds over time. The collected data may also be time and/or date stamped. For example, the first microphone 106 records the breathing sounds at the nose 110 of the user 104. In other examples, the first microphone 106 records breathing sounds at a mouth 112 of the user and/or at the nose 110 and the mouth 112 of the user 104. For a healthy subject, breathing sound frequencies may range from 60 Hz to 1,000 Hz, with most power of the corresponding signal data falling between 60 Hz and 600 Hz. In some examples, the first microphone 106 captures (e.g., records) other sound data such as a sounds associated with the user's voice, environmental sounds, etc. As disclosed herein, parameters for the collection of sounds by the first microphone 106 can be defined by one or more rules (e.g., user settings) with respect to, for example, the duration for which the sound(s) are to be recorded (e.g., always recording when the user 104 is wears the HMD 102, not always on when the user is wearing the HMD 102). The example HMD 102 can include additional microphones to collect breathing sounds generated by the user 104.

The example system 100 of FIG. 1 includes one or more processors to access breathing sound data 114 collected by the first microphone 106, process the breathing sound data 114 collected by the first microphone 106, and/or generate one or more outputs based on the processing of the breathing sound data 114. For example, as illustrated in FIG. 1, a processor 116 is coupled to (e.g., mounted to, carried by) the HMD 102 (e.g., the frame 107). In other examples, the processor 116 is separate from the HMD 102. In some examples, the processor 116 (e.g., the first processor) is a digital signal processor.

The first microphone 106 may transmit the breathing sound data 114 to the first processor 116 using any past, present, or future communication protocol. In some examples, the first microphone 106 transmits the breathing sound data 114 to the first processor 116 in substantially real-time as the breathing sound data 114 is generated. In other examples, the first microphone 106 transmits the breathing sound data 114 to the first processor 116 at a later time (e.g., based on one or more settings such as a preset time of transmission, availability of Wi-Fi, etc.).

In some examples, the first processor 116 converts the breathing sound data 114 collected by the first microphone 106 from analog to digital data (if the first microphone 106 does not provide a digital output). The breathing sound data 114 collected by the first microphone 106 can be stored in a memory or buffer of the first processor 116 as, for example, an audio file (e.g., a WAV file).

The example HMD 102 of FIG. 1 includes a second microphone 118 coupled (e.g., mounted) to the HMD 102. As illustrated in FIG. 1, in some examples, the second microphone 118 is coupled to the frame 107 of the HMD 102 such that the second microphone 118 is spaced apart from the nose 110 and/or mouth 112 of the user 104 and/or the first microphone 106. In examples where the first microphone 106 is coupled proximate to the nose bridge of the HMD 102 (e.g., the eyeglasses), the second microphone 118 can be coupled proximate to, for example, an earpiece of the HMD 102. The second microphone 118 can be coupled to the HMD 102 at other locations than illustrated in FIG. 1.

The second microphone 118 of FIG. 1 collects (e.g., records) ambient sounds (e.g., noise) from an environment in which the user 104 is located when the user 104 is wearing the HMD 102 over time. In some examples, the second microphone 118 collects the ambient sounds at substantially the same time that the first microphone 106 collects the breathing sounds. For example, if the user 104 is wearing the HMD 102 while the user 104 is taking a walk at a park, the first microphone records the user's breathing sounds and the second microphone 118 records ambient sounds such as other people talking, nearby traffic, the wind, etc. In some examples, the second microphone 118 records sounds generated by the user other than breathing such as the user's voice, coughing by the user, etc. As disclosed herein, parameters concerning the collection of ambient sounds by the second microphone 118 (e.g., a duration for which the sound(s) are to be recorded) can be based on one or more rules (e.g., user settings). The HMD 102 can include additional microphones to collect ambient sounds from the environment in which the user 104 is located. In other examples, the HMD 102 only includes the first microphone 106 to collect breathing sounds.

In the example system 100 of FIG. 1, the second microphone 118 transmits environmental noise or ambient sound data 120 to the first processor 116. The second microphone 118 may transmit the ambient sound data 120 to the first processor 116 using any past, present, or future communication protocol. The second microphone 118 may transmit the ambient sound data 120 to the first processor 116 in substantially real-time as the ambient sound data 120 is generated or at a later time. In some examples, the second microphone 118 is a digital microphone that provides a digital output. In other examples, the first processor 116 converts the ambient sound data 120 from analog to digital data. The ambient sound data 120 collected by the second microphone 118 can be stored in the memory or buffer of the first processor 116 as, for example, an audio file (e.g., a WAV file).

In the example of FIG. 1, the breathing sound data 114 is processed by a breathing pattern detector 122 of the first processor 116. The breathing pattern detector 122 of the first processor 116 serves to process the breathing sound data 114 collected by the first microphone 106 to detect the breathing pattern for the user 104. As disclosed herein, the first microphone 106 may capture other noises in addition to the breathing sounds associated with inhalation and exhalation by the user 104, such as the user's voice, other lung sounds such as wheezing which can appear at frequencies above 2,000 Hz, and/or other sounds from the environment. As also disclosed herein, the second microphone 118 collects the ambient noise data 120 as substantially the same time that the first microphone 106 is collecting sound data. To facilitate synchronization of the two data sets, all sound data may be time and/or date stamped as it is collected by the first and/or second microphones 106, 118. The example breathing pattern detector 122 of FIG. 1 modifies (e.g., filters) the breathing sound data 114 to remove or substantially remove environmental noise data from the breathing sound data 114 that may have been captured by the first microphone 106. In the example of FIG. 1, the breathing pattern detector 122 deducts (e.g., subtracts) the ambient sound data 120 collected by the second microphone 118 from the breathing sound data 114 to remove noise from the breathing sound data 114.

The breathing pattern detector 122 further filters (e.g., bandpass filters) the remaining breathing sound signal data to remove high and/or low frequencies and to pass the frequency band containing most of the power of the signal data corresponding to breathing sounds generated during inhalation and exhalation. For example, the breathing pattern detector 122 may filter out frequencies less than 100 Hz, which may contain heart and/or muscle sounds. The example breathing pattern detector 122 processes the filtered breathing sound data to detect a breathing pattern of the user 104 and to generate breathing pattern data 126. In some examples, the breathing pattern detector 122 processes the filtered breathing sound data by downsampling (e.g., reducing a sampling rate of) the filtered breathing sound data and calculating an envelope for the filtered breathing sound data.

In some examples, the breathing pattern detector 122 generates the breathing pattern data 126 based on a number of peaks in the breathing sound data 114 over time, where the peaks are indicative of inhalations and exhalations. Additionally or alternatively, the example breathing pattern detector 122 of FIG. 1 can detect the breathing pattern based on other characteristics of the breathing sound data, such as amplitudes of the peaks in the data, durations between the peaks, etc. Based on the signal data characteristics, the breathing pattern detector 122 can generate metrics indicative of the user's breathing pattern, such as breathing rate.

In the example system 100 of FIG. 1, the breathing pattern detector 122 (e.g., a digital signal processor) transmits the breathing pattern data 126 to a second processor 128 (e.g., a microcontroller) for storage and/or further analysis. The second processor 128 can be coupled to (e.g., mounted to, carried by) the HMD 102 (e.g., the frame 107). In other examples, the second processor 128 is separate from the HMD 102. In some examples, the HMD 102 only includes the second processor 128 and the breathing pattern detector 122 is implemented by the second processor 128.

The example second processor 128 of FIG. 1 writes the breathing pattern data 126 to a memory. In some examples, the on-board second processor 128 transmits the breathing pattern data 126 to a user device 130 different than the HMD 102. The user device 130 can include, for example, a smartphone, a personal computer, another wearable device (e.g., a wearable fitness monitor), etc. In some examples, the second processor 128 of the HMD 102 and the user device 130 are communicatively coupled via one or more wired connections (e.g., a cable) or wireless connections (e.g., Wi-Fi or Bluetooth connections).

In the example of FIG. 1, the breathing pattern data 126 is processed by a breathing pattern analyzer 132 to generate one or more outputs based on the breathing pattern data 126. The example breathing pattern analyzer 132 can be implemented by the first processor 116 or the second processor 128. In some examples, one or more components of the example breathing pattern analyzer 132 are implemented by one of the first processor 116 or the second processor 128 and one or more other components are implemented by the other of the first processor 116 or the second processor 128. One or more of the processors 116, 128 maybe located remotely from the HMD 102 (e.g., at the user device 130). In some examples, both processors 116, 128 are carried by the HMD 102. In some examples, one or more of the components of the breathing pattern analyzer 132 are implemented by the first processor 116 and/or second processor 128 carried by the HMD 102 and one or more other components are implemented by another processor at the user device 130.

In the example of FIG. 1, the breathing pattern analyzer 132 analyzes the breathing pattern data 126 to generate output(s) including notification(s) and/or alert(s) with respect to, for example, breathing performance metrics (e.g., breathing rate, breathing capacity) and/or health conditions associated with the breathing performance metrics such as stress levels. The breathing pattern analyzer 132 analyzes the breathing pattern data 126 and generates the output(s) based one or more predefined rules. The output(s) can be presented via the user device 130 and/or the HMD 102 as visual, audio, and/or tactile alert(s) and/or notification(s).

In some examples, the breathing pattern analyzer 132 stores one or more rules that define user control settings for the HMD 102. For example, the rule(s) can define durations of time that the first microphone 106 and the second microphone 118 are to collect sound data, decibel and/or frequency thresholds for the collection of sounds by the respective microphones 106, 118, etc. Thus, in some examples, the breathing pattern analyzer 132 can be used to control one or more components of the HMD 102 (e.g., via second processor 128 of the HMD 102 and/or the user device 130).

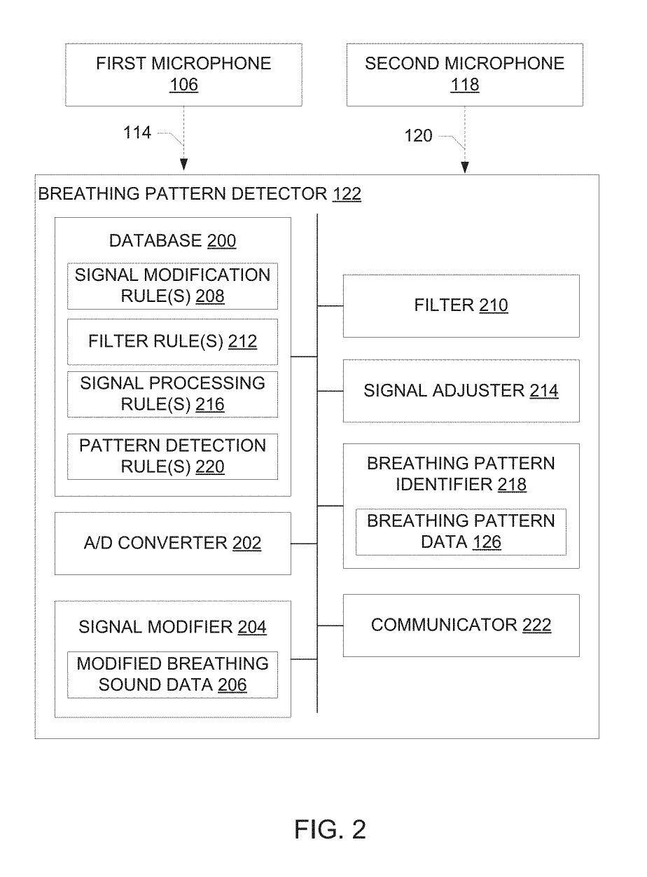

FIG. 2 is a block diagram of an example implementation of the example breathing pattern detector 122 of FIG. 1. As mentioned above, the example breathing pattern detector 122 is constructed to detect one or more breathing patterns of a user (e.g., the user 104 of FIG. 1) based on the breathing sounds collected via the first microphone 106 of the HMD 102 of FIG. 1. In the example of FIG. 2, the breathing pattern detector 122 is implemented by the first processor 116 (e.g., a digital signal processor) of the HMD 102. In other examples, the breathing pattern detector 122 is implemented by the second processor 128 (e.g., a microcontroller) and/or a combination of the first processor 116 and the second processor 128.

The example breathing pattern detector 122 of FIG. 2 includes a database 200. In other examples, the database 200 is located external to the breathing pattern detector 122 in a location accessible to the detector. The database 200 can be stored in one or more memories. The memory/memories storing the databases may be on-board the first processor 116 (e.g., one or more memories of a digital signal processor for storing instructions and data) and/or may be external to the first processor 116.

As disclosed herein, the breathing sound data 114 collected (e.g., recorded) by the first microphone 106 as the user 104 breathes is transmitted to the breathing pattern detector 122. This transmission may be substantially in real time (e.g., as the data is gathered), periodically (e.g., every five seconds), and/or may be aperiodic (e.g., based on factor(s) such as an amount of data collected, memory storage capacity usage, detection that the user exercising (e.g., based on motion sensors), etc.). As also disclosed herein, the ambient sound data 120 collected (e.g., recorded) by the second microphone 118 is also transmitted to the breathing pattern detector 122. This transmission may be substantially in real time, periodic, or aperiodic. In the illustrated example, the database 200 provides means for storing the breathing sound data 114 and the ambient sound data 120. In some examples, the breathing sound data 114 and/or the ambient sound data 120 are stored in the database 200 temporarily and/or are discarded or overwritten as additional breathing sound data 114 and/or ambient sound data 120 are generated and received by the breathing pattern detector 122 over time.

In some examples, the first microphone 106 and/or the second microphone 118 are digital microphones that provide digital signal outputs. In other examples, the breathing pattern detector 122 includes an analog-to-digital (A/D) converter 202 that provides means for converting the analog breathing sound data 114 to digital signal data and/or converting the analog ambient sound data 120 to digital signal data for analysis by the example breathing pattern detector 122.

As disclosed herein, in some examples, the breathing sound data 114 may include noise captured by the first microphone 106 that is not associated with breathing sounds, such as the user's voice, environmental noises, etc. The example breathing pattern detector 122 of FIG. 2 substantially reduces (e.g., removes) noise in the breathing sound data 114 so that the noise does not interfere with the detection of the breathing pattern. The example breathing pattern detector 122 includes a signal modifier 204. In the illustrated example, the signal modifier 204 provides means for modifying the breathing sound data 114 based on one or more signal modification rules 208 by removing noise from the breathing sound data 114 (e.g., environmental noise, other noises generated by the user 104 such as the user's voice) to generate modified breathing sound data. The rule(s) 208 instruct the signal modifier 204 to perform one or more operations on the signal data to substantially cancel noise from the breathing sound data 114 collected by the first microphone 106. The rule(s) 208 can be defined by user input(s) received by the breathing pattern detector 122. The rule(s) 208 may be stored in the database 200 or in another storage location accessible to the signal modifier 204.

In the example of FIG. 2, the signal modifier 204 deducts or subtracts the ambient sound data 120 from the breathing sound data 114 based on the signal modification rule(s) 208 to generate modified breathing sound data 206. The modified breathing sound data 206 (e.g., the breathing sound data 114 remaining after the subtraction of the ambient sound data 120) represents the breathing sounds generated by the user 104 without noise data that may have been captured by the first microphone 106. Thus, the signal modifier 204 substantially reduces or eliminates environmental noise from the breathing sound data 114. In some examples, the signal modifier 204 aligns and/or correlates (e.g., based on time) the breathing sound data 114 and the ambient sound data 120 before modifying the breathing sound data 114 to remove background/environmental noise.

The example signal modifier 204 can perform other operations to modify the breathing sound data 114. For example, the signal modifier 204 can convert time domain audio data into the frequency spectrum (e.g., via Fast Fourier processing (FFT)) for spectral analysis.

The example breathing pattern detector 122 of FIG. 2 includes a filter 210 (e.g., a band pass filter). In the illustrated example, the filter 210 provides means for further filtering the modified breathing sound data 206. For example, the filter 210 filters the modified breathing sound data 206 to remove low frequencies associated with, for example, heart and/or muscle sounds (e.g., frequencies less than 100 Hz) and/or to remove high frequencies that may be associated with, for example, wheezing or coughing (e.g., frequencies above 1,000 Hz). The filter 210 may pass frequencies within a frequency band known to contain most of the power for the breathing signal data (e.g., 400 Hz to 600 Hz). The frequencies passed or filtered by the filter 210 of FIG. 2 can be defined by filter rule(s) 212 stored in the database 200. In some examples, the filter rule(s) 212 are based on user characteristics such as age, health conditions, etc. that may affect frequencies of the user's breathing sounds (e.g., whether the user breathes softly or loudly, etc.).

The example breathing pattern detector 122 of FIG. 2 includes a signal adjuster 214. In the illustrated example, the signal adjuster 214 provides means for processing the modified (e.g., filtered) breathing sound data 206. The signal adjuster 214 processes the modified breathing sound data 206 based on signal processing rule(s) 216. For example, the signal adjuster 214 can downsample or reduce the sampling rate of the modified breathing sound data 206 to reduce a size of the data analyzed by the breathing pattern detector 122. In some examples, the signal adjuster 214 reduces the sampling rate to increase an efficiency of the breathing pattern detector 122 in detecting the breathing pattern in substantially real-time as the breathing sound data 114 is received at the breathing pattern detector 122. In some examples, the signal adjuster 214 divides the signal data into frames to be analyzed by the breathing pattern detector 122. In some examples, the signal adjuster 214 calculates an envelope (e.g., a root-mean-square envelope) for the modified breathing sound data 206 based on the signal processing rule(s) 216. The envelope calculated by the signal adjuster 214 can indicate changes in the breathing sounds generated by the user 104 over time, such as changes in amplitude.

The example breathing pattern detector 122 of FIG. 2 includes a breathing pattern identifier 218. In the illustrated example, the breathing pattern identifier 218 provides means for analyzing the breathing sound data processed by the signal modifier 204, the filter 210, and/or the signal adjuster 214 to identify the breathing pattern(s) and generate the breathing pattern data 126. In the example of FIG. 2, the breathing pattern identifier 218 identifies the breathing pattern based on one or more pattern detection rule(s) 220. In the example of FIG. 2, the pattern detection rules(s) 220 are stored in the database 200.

For example, the breathing pattern identifier 218 can detect peaks (e.g., inflection points) in the modified breathing sound data 206 processed by the signal adjuster 214. In some examples, the breathing pattern identifier 218 identifies the peaks based on changes in amplitudes represented by the signal envelope calculated by the signal adjuster 214. The breathing pattern identifier 218 can classify the peaks as associated with inhalation or exhalation based on the pattern detection rule(s) 220. For example, the breathing pattern identifier 218 can classify the peaks as associated with inhalation or exhalation based on amplitude thresholds defined by the pattern detection rule(s) 220.

Based on the classification of the peaks and the pattern detection rule(s) 220, the breathing pattern identifier 218 of this example detects the breathing pattern(s). For example, the breathing pattern identifier 218 can determine the number of inhalation peaks and/or exhalation peaks within a period of time and compare the number of peaks to known breathing pattern peak thresholds defined by the rule(s) 220. The breathing pattern peak thresholds can include known numbers of inhalation peaks and/or exhalation peaks associated with breathing during different activities such as running or sitting quietly for the user 104 and/or other users and/or as a result of different health conditions (e.g., asthma). The breathing pattern identifier 218 can generate the breathing pattern data 126 based on classifications of the breathing sound signal data in view of reference threshold(s).

In some examples, the breathing pattern identifier 218 determines that the breathing pattern is irregular as compared to reference data for substantially normal (e.g., regular) breathing as defined by the pattern detection rule(s) 220 for the user 104 and/or other users. For example, the breathing pattern identifier 218 can detect irregularities in the breathing sound data, such as varying amplitudes of the peaks, changes in durations between inhalation peaks, etc. from breathing cycle to breathing cycle. In such examples, the breathing pattern identifier 218 generates the breathing pattern data 126 classifying the breathing pattern as irregular.

As another, the example breathing pattern identifier 218 can generate the breathing pattern data 126 by calculating one more metrics based on one or more features of the breathing sound signal data, such as peak amplitude, frequency, duration of time between peaks, distances between peaks, etc. For example, the example breathing pattern can calculate a number of breaths per minute and generate the breathing pattern data 126 based on the breathing rate. As another example, the breathing pattern identifier 218 can calculate or estimate tidal volume, or a volume of air displaced between inhalation and exhalation, based on the number of peaks, frequency of the peaks, and/or average tidal volumes based on body mass, age, etc. of the user 104. As another example, the breathing pattern identifier 218 can generate metrics indicating durations of inhalation and/or durations of exhalation based on characteristics of the peaks in the signal data.

The example breathing pattern detector 122 of FIG. 2 includes a communicator 222 (e.g., a transmitter, a receiver, a transceiver, a modem, etc.). In examples where the breathing pattern detector 122 is implemented by, for example, a digital signal processor, the communicator 222 provides means for transmitting the breathing pattern data 126 to, for example the second processor 128 of the HMD 102 for storage and/or further analysis. For instance, the communicator 222 can transmit the breathing pattern data 126 via wireless and/or wired connections between the first processor 116 and the second processor 128 at, for example, the HMD 102.

While an example manner of implementing the example breathing pattern detector 122 is illustrated in FIG. 2, one or more of the elements, processes and/or devices illustrated in FIG. 2 may be combined, divided, re-arranged, omitted, eliminated and/or implemented in any other way. Further, the example database 200, the example A/D converter 202, the example signal modifier 204, the example filter 210, the example signal adjuster 214, the example breathing pattern identifier 218, the example communicator 222 and/or, more generally, the example breathing pattern detector 122 of FIG. 2 may be implemented by hardware, software, firmware and/or any combination of hardware, software and/or firmware. Thus, for example, any of the example database 200, the example A/D converter 202, the example signal modifier 204, the example filter 210, the example signal adjuster 214, the example breathing pattern identifier 218, the example communicator 222 and/or, more generally, the example breathing pattern detector 122 of FIG. 2 could be implemented by one or more analog or digital circuit(s), logic circuits, programmable processor(s), application specific integrated circuit(s) (ASIC(s)), programmable logic device(s) (PLD(s)) and/or field programmable logic device(s) (FPLD(s)). When reading any of the apparatus or system claims of this patent to cover a purely software and/or firmware implementation, at least one of the example database 200, the example A/D converter 202, the example signal modifier 204, the example filter 210, the example signal adjuster 214, the example breathing pattern identifier 218, the example communicator 222 and/or, more generally, the example breathing pattern detector 122 of FIG. 2 is/are hereby expressly defined to include a non-transitory computer readable storage device or storage disk such as a memory, a digital versatile disk (DVD), a compact disk (CD), a Blu-ray disk, etc. including the software and/or firmware. Further still, the example breathing pattern detector 122 of FIGS. 1 and 2 may include one or more elements, processes and/or devices in addition to, or instead of, those illustrated in FIGS. 1 and 2, and/or may include more than one of any or all of the illustrated elements, processes and devices.

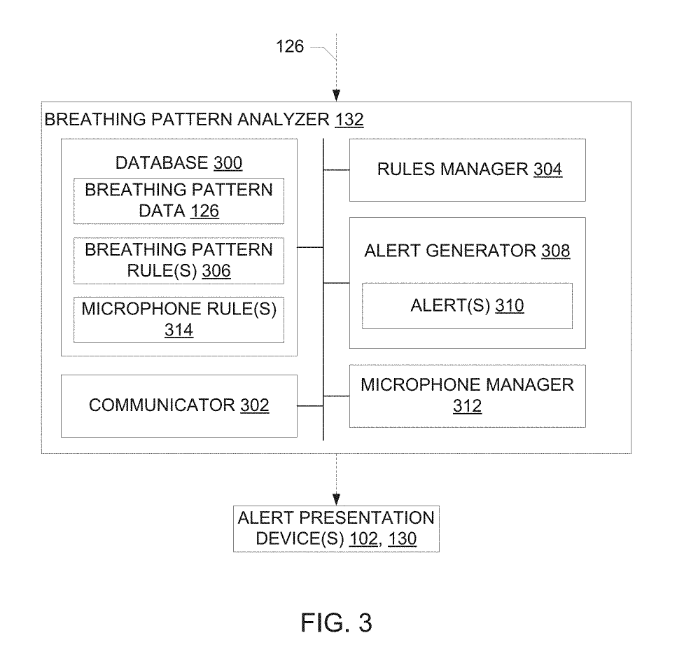

FIG. 3 is a block diagram of an example implementation of the example breathing pattern analyzer 132 of FIG. 1. As mentioned above, the example breathing pattern analyzer 132 is constructed to analyze the breathing pattern data 126 generated by the example breathing pattern detector 122 of FIGS. 1 and 2 to generate one or more outputs (e.g., alert(s), notification(s)). In the example of FIG. 3, the breathing pattern analyzer 132 is implemented by the second processor 128 (e.g., a microcontroller). In some examples, the second processor 128 is carried by the HMD 102. In other examples, the second processor 128 is located at the user device 130. In some examples, one or more of the components of the breathing pattern analyzer 132 are implemented by the second processor 128 carried by the HMD 102 and one or more other components are implemented by another processor at the user device 130. In other examples, one or more of the components of the breathing pattern analyzer 132 are implemented by the first processor 116 (e.g., a digital signal processor).

The breathing pattern analyzer 132 of this example includes a database 300. In other examples, the database 300 is located external to the breathing pattern analyzer 132 in a location accessible to the analyzer. As disclosed herein, the breathing pattern analyzer 132 receives the breathing pattern data 126 from the breathing pattern detector 122 (e.g., via communication between the first processor 116 and the second processor 128). In the illustrated example, the database 300 provides means for storing the breathing pattern data 126 generated by the breathing pattern detector 122. In some examples, the database 300 stores the breathing pattern data 126 over time to generate historical breathing pattern data.

The example breathing pattern analyzer 132 includes a communicator 302 (e.g., a transmitter, a receiver, a transceiver, a modem, etc.). As disclosed herein, in some examples, the breathing pattern data 126 is transmitted from the second processor 128 to the user device 130. In some such examples, the second processor 128 provides for storage (e.g., temporary storage) of the breathing pattern data 126 received from the breathing pattern detector 122 of FIG. 2 and the breathing pattern data 126 is analyzed at the user device 130.

The example breathing pattern analyzer 132 includes a rules manager 304. In the illustrated example, the rules manager 304 provides means for applying one or more breathing pattern rule(s) 306 to the breathing pattern data 126 to generate one or more outputs, such as alert(s) or notification(s) that provide for monitoring of the user's breathing.

In the example of FIG. 3, the breathing pattern rule(s) 306 can be defined by one or more user inputs. The breathing pattern rule(s) 306 can include, for example, thresholds and/or criteria for the breathing pattern data 126 (e.g., the breathing metrics) that trigger alert(s). The rules manager 304 applies the breathing pattern rule(s) 306 to determine if, for example, the breathing pattern data 126 satisfies a threshold (e.g., exceeding the threshold, failing to meet the threshold, equaling the threshold depending on the context and implementation). For example, the breathing pattern rule(s) 306 can indicate that an alert should be generated if the breathing rate exceeds a threshold breathing rate for the user 104 based on one more characteristics of the user 104 and/or other users (e.g., fitness level). In some examples, the breathing pattern rule(s) 306 include a rule indicating that an alert is to be generated if there is a change detected in the breathing pattern data 126 over a threshold period of time (e.g., 1 minute, 15 seconds, etc.) and/or relative to historical breathing pattern data stored in the database 300 (e.g., more than a threshold increase in breathing rate over time). In some examples, the breathing pattern rule(s) 306 includes a rule indicating that an alert is to be generated if the breathing pattern data 126 is indicative of irregular breathing patterns associated with, for example, hyperventilation, an asthma attack, etc. that are included as reference data in the breathing pattern rule(s) 306. In other examples, the rule(s) 306 indicate that the breathing pattern data 126 (e.g., breathing rate, inhalation and exhalation duration data) should be always provided to the user while the user 104 is wearing the HMD 102.

The example rules manager 304 of FIG. 3 applies the breathing pattern rule(s) 306 to the breathing pattern data 126. The rules manager 304 determines if, for example, the breathing pattern data 126 satisfies one or more threshold(s) and/or criteria defined by the rule(s) 306. Based on the analysis, the rules manager 304 determines whether alert(s) or notification(s) should be generated.

The example breathing pattern analyzer 132 of FIG. 3 includes an alert generator 308. In the illustrated example, the alert generator 308 provides means for generating one or more alert(s) 310 for output by the breathing pattern analyzer 132 based on the analysis of the breathing pattern data 126 by the rules manager 304. The alert(s) 310 can include warnings, notifications, etc. for presentation via the HMD 102 and/or the user device 130. The alert(s) 310 can be presented in audio, visual, and/or tactile formats. For example, the alert(s) 310 can include breathing rate data and/or breathing efficiency metrics for display via a screen of the user device 130 that is updated in substantially real-time based on the analysis of the breathing sound data 114 by breathing pattern detector 122 and the breathing pattern analyzer 132. As another example, the alert(s) 310 can include a warning that the user should reduce activity and/or seek medical attention if the analysis of the breathing sound data 114 indicates potential health conditions.

In some examples, the alert generator 308 only generates the alert(s) 310 if one or more conditions (e.g., predefined conditions) are met. For example, the alert generator 308 may generate the alert(s) 310 in substantially real-time as breathing pattern data 126 is analyzed by the rules manager 304. In other such examples, the alert generator 308 generates the alert(s) 310 when there is no further breathing pattern data 126 for analysis by the rules manager 304.

In the example of FIG. 3, the communicator 302 communicates with one or more alert presentation devices, which can include the user device 130 and/or the HMD 102 and/or be carried by the HMD 102, to deliver the alert(s) 310 for presentation, storage, etc.

The example breathing pattern analyzer 132 of FIG. 3 also manages the collection of sound data by the first and/or second microphones 106, 118 of the HMD 102. To this end, the example breathing pattern analyzer 132 includes a microphone manager 312. In the illustrated example, the microphone manager 312 provides means for controlling the collection of the breathing sound data 114 by the first microphone 106 and/or the collection of the ambient sound data 120 by the second microphone 118. The example microphone manager 312 of FIG. 3 applies one or more microphone rule(s) 314 to control the microphone(s) 106, 118 (e.g., rules determining how often the microphones are active, etc.).

The microphone rule(s) 314 can be defined by one or more user inputs and/or stored in the database 300 or another location. In some examples, the microphone rule(s) 314 instruct that the first microphone 106 and/or the second microphone 118 should be “always on” in that they always collect sound data (e.g., when the user 104 is wearing the HMD 102). In other examples, the microphone rule(s) 314 instruct that the first microphone 106 and/or the second microphone 118 only record sound(s) if the sound(s) surpass threshold amplitude levels. In some examples, the microphone rule(s) 314 define separate threshold levels for the first microphone 106 and the second microphone 118 so that the first microphone 106 captures, for example, lower frequency breathing sounds as compared to environmental noises captured by the second microphone 118. In other examples, the threshold(s) for the first microphone 106 and/or the second microphone 118 is based on one or more other characteristics of the breathing sounds and/or the ambient sounds, such as pattern(s) of the sound(s) and/or duration(s) of the sound(s). In such examples, the first microphone 106 and/or the second microphone 118 only collect sound data 114, 120 if the threshold(s) defined by the rule(s) 314 are met (i.e., the microphone(s) 106, 118 are not “always on” but instead are activated for audio collection only when certain conditions are met (e.g., time of day, the HMD 102 being worn as detected by a sensor, etc.).

The microphone rule(s) 314 can be defined by a third party and/or the user 104 of the HMD 102. In some examples, the microphone rule(s) 314 are updated by the user 104 via the HMD 102 and/or the user device 130. The microphone manager 312 communicates with the communicator 302 to deliver instructions to the first microphone 106 and/or the second microphone 118 with respect to the collection of sound data by each microphone at the HMD 102.

While an example manner of implementing the example breathing pattern analyzer 132 is illustrated in FIG. 3, one or more of the elements, processes and/or devices illustrated in FIG. 3 may be combined, divided, re-arranged, omitted, eliminated and/or implemented in any other way. Further, the example database 300, the example communicator 302, the example rules manager 304, the example alert generator 308, the example microphone manager 312 and/or, more generally, the example breathing pattern analyzer 132 of FIG. 3 may be implemented by hardware, software, firmware and/or any combination of hardware, software and/or firmware. Thus, for example, any of the example database 300, the example communicator 302, the example rules manager 304, the example alert generator 308, the example microphone manager 312 and/or, more generally, the example breathing pattern analyzer 132 of FIG. 3 could be implemented by one or more analog or digital circuit(s), logic circuits, programmable processor(s), application specific integrated circuit(s) (ASIC(s)), programmable logic device(s) (PLD(s)) and/or field programmable logic device(s) (FPLD(s)). When reading any of the apparatus or system claims of this patent to cover a purely software and/or firmware implementation, at least one of the example database 300, the example communicator 302, the example rules manager 304, the example alert generator 308, the example microphone manager 312 and/or, more generally, the example breathing pattern analyzer 132 of FIG. 3 is/are hereby expressly defined to include a non-transitory computer readable storage device or storage disk such as a memory, a digital versatile disk (DVD), a compact disk (CD), a Blu-ray disk, etc. including the software and/or firmware. Further still, the example breathing pattern analyzer 132 of FIGS. 1 and 3 may include one or more elements, processes and/or devices in addition to, or instead of, those illustrated in FIGS. 1 and 3, and/or may include more than one of any or all of the illustrated elements, processes and devices.

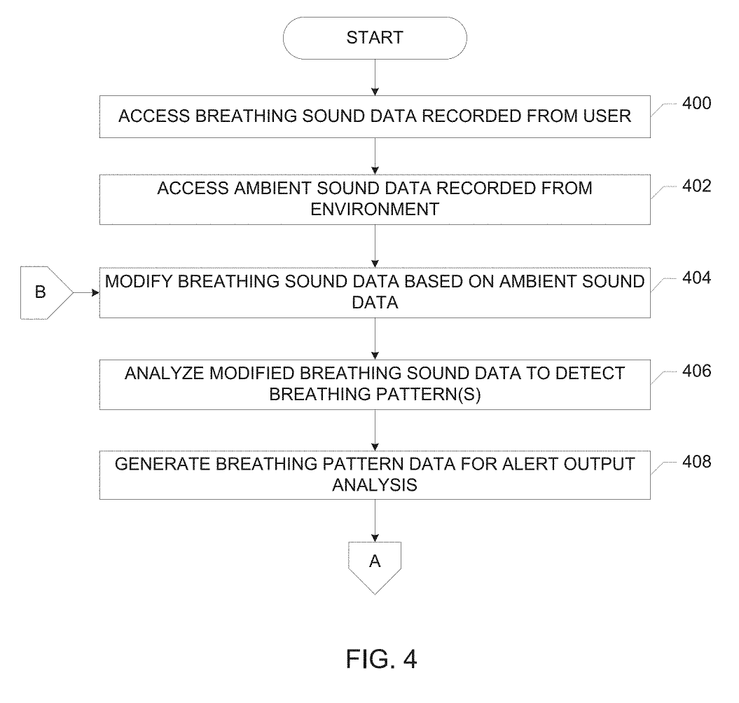

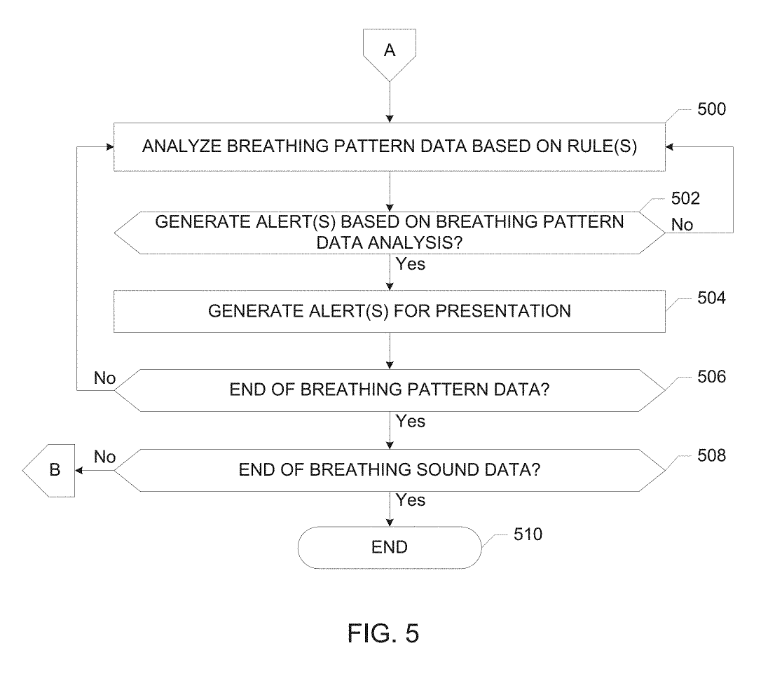

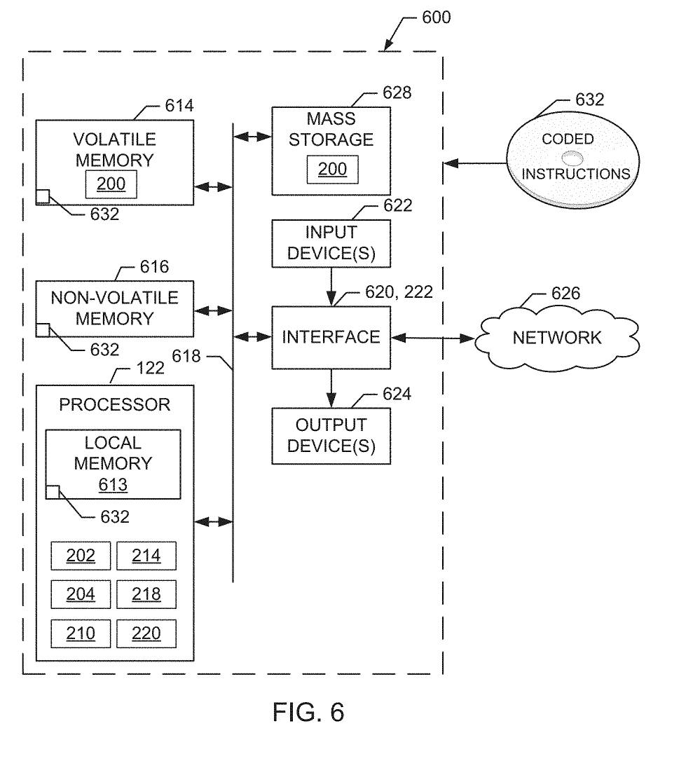

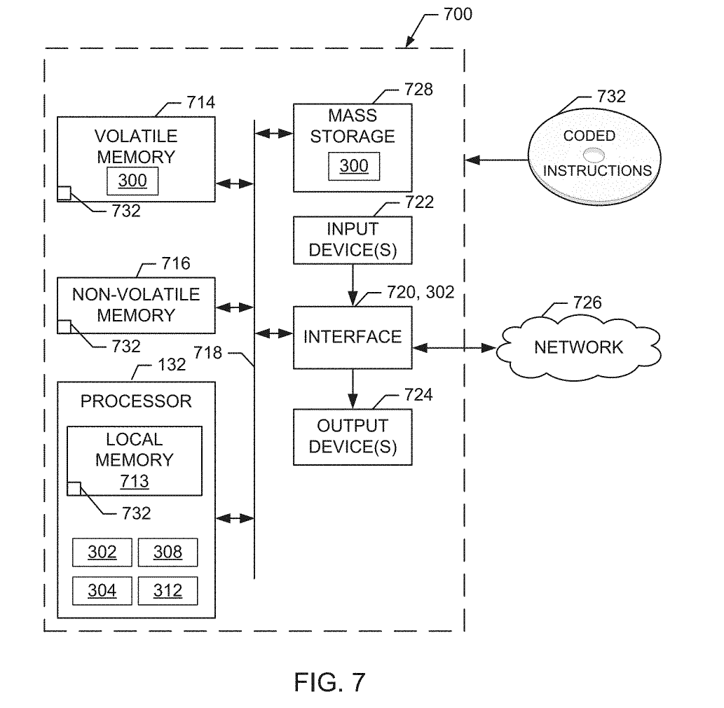

Flowcharts representative of example machine readable instructions for implementing the example system 100 and/or components thereof illustrated in of FIGS. 1, 2, and/or 3 are shown in FIGS. 4 and 5. In these examples, the machine readable instructions comprise a program for execution by one or more processors such as the processor(s) 122, 132 shown in the example processor platforms 600, 700 discussed below in connection with FIGS. 6 and 7. The program may be embodied in software stored on a non-transitory computer readable storage medium such as a CD-ROM, a floppy disk, a hard drive, a digital versatile disk (DVD), a Blu-ray disk, or a memory associated with the processor(s) 122, 132 but the entire program and/or parts thereof could alternatively be executed by device(s) other than the processor(s) 122, 132 and/or embodied in firmware or dedicated hardware. Further, although the example program is described with reference to the flowcharts illustrated in FIGS. 4 and 5, many other methods of implementing the example system 100 and/or components thereof illustrated in of FIGS. 1, 2, and/or 3 may alternatively be used. For example, the order of execution of the blocks may be changed, and/or some of the blocks described may be changed, eliminated, or combined. Additionally or alternatively, any or all of the blocks may be implemented by one or more hardware circuits (e.g., discrete and/or integrated analog and/or digital circuitry, a Field Programmable Gate Array (FPGA), an Application Specific Integrated circuit (ASIC), a comparator, an operational-amplifier (op-amp), a logic circuit, etc.) structured to perform the corresponding operation without executing software or firmware.

As mentioned above, the example processes of FIGS. 4 and 5 may be implemented using coded instructions (e.g., computer and/or machine readable instructions) stored on a non-transitory computer and/or machine readable medium such as a hard disk drive, a flash memory, a read-only memory, a compact disk, a digital versatile disk, a cache, a random-access memory and/or any other storage device or storage disk in which information is stored for any duration (e.g., for extended time periods, permanently, for brief instances, for temporarily buffering, and/or for caching of the information). As used herein, the term non-transitory computer readable medium is expressly defined to include any type of computer readable storage device and/or storage disk and to exclude propagating signals and to exclude transmission media. “Including” and “comprising” (and all forms and tenses thereof) are used herein to be open ended terms. Thus, whenever a claim lists anything following any form of “include” or “comprise” (e.g., comprises, includes, comprising, including, etc.), it is to be understood that additional elements, terms, etc. may be present without falling outside the scope of the corresponding claim. As used herein, when the phrase “at least” is used as the transition term in a preamble of a claim, it is open-ended in the same manner as the term “comprising” and “including” are open ended.

FIG. 4 is a flowchart representative of example machine-readable instructions that, when executed, cause the example breathing pattern detector 122 of FIGS. 1 and/or 2 to detect breathing pattern(s) by a user (e.g., the user 104 of FIG. 1) based on breathing sound(s) generated by the user during inhalation and exhalation. In the example of FIG. 4, the breathing sound(s) can be collected (e.g., recorded) by the first microphone 106 of the HMD 102 of FIG. 1. In the example of FIG. 4, the ambient sound(s) can be collected by the second microphone 118 of the HMD 102 of FIG. 1. The example instructions of FIG. 4 can be executed by, for example, the first processor 116 of FIG. 1 to implement the breathing pattern detector 122 of FIGS. 1 and/or 2.

The example signal modifier 204 of the breathing pattern detector 122 of FIG. 2 accesses the breathing sound data 114 generated over time by the user 104 wearing the HMD 102 including the first microphone 106 (block 400). In some examples, the breathing sound data 114 includes digital signal data generated by the digital first microphone 106. In other examples, the breathing sound data 114 is converted by the A/D converter 202 to digital signal data.

The example signal modifier 204 of the breathing pattern detector 122 accesses the ambient sound data 120 generated over time based on, for example, noises in an environment in which the user 104 is located while wearing the HMD 102 including the second microphone 118 (block 402). In the example of FIG. 3, the ambient noise data is collected by the second microphone 118 at substantially the same time that the breathing sound data 114 is collected by the first microphone 106 to facilitate synchronization of the data sets. In some examples, the ambient sound data 120 includes digital signal data generated by the digital second microphone 118. In other examples, the ambient sound data 120 is converted by the A/D converter 202 to digital signal data.

The example signal modifier 204 modifies the breathing sound data 114 based on the ambient sound data 120 to substantially reduce (e.g., remove) noise in the breathing sound data 114 due to, for example, sounds in the environment in which the user 104 is located and that are captured by the first microphone 106 (block 404). For example, the signal modifier 204 deducts or subtracts the ambient sound data 120 from the breathing sound data 114 to account for environmental noises and/or other noises generated by the user (e.g., wheezing, the user's voice) that appear in the breathing sound data 114. In some examples, the signal modifier 204 aligns or correlates the breathing sound data 114 and the ambient noise data 120 (e.g., based on time) prior to the subtraction. The signal modifier 204 generates modified breathing sound data 206 that includes the breathing sound data without and/or with substantially reduced noise levels.

The breathing pattern detector 122 can perform other operations to process the breathing sound data 206. For example, the signal modifier 204 can convert the breathing sound data 206 to the frequency domain. The filter 210 of the breathing pattern detector 122 can apply a bandpass filter to filter out low and/or high frequencies associated with other noises, such as heart sounds, coughing noises, etc.

The breathing pattern detector 122 analyzes the modified (e.g., filtered) breathing sound data 206 to detect the breathing pattern(s) represented by the data (block 406). For example, the signal adjuster 214 of the breathing pattern detector 122 calculates an envelope for the breathing sound data 206 that is used to identify peaks and corresponding amplitudes in the signal data and/or apply other operations based on the signal processing rule(s) 216. In this example, the breathing pattern identifier 218 detects peaks in the breathing sound data 114 indicative of inhalation and exhalations. The breathing pattern identifier 218 calculates one or more breathing metrics (e.g., breathing rate) based on the characteristics of the peaks, such as amplitude, frequency, duration, etc. In other examples, the breathing pattern identifier 218 detects the breathing pattern(s) by comparing the breathing sound data to reference data defined by the pattern detection rule(s) 220.

The breathing pattern identifier 218 generates the breathing pattern data 126 based on the analysis of the breathing sound data 206 (block 408). The breathing pattern data 126 can include, for example, breathing metrics that characterize the breathing pattern (e.g., breathing rate, tidal volume) and/or other classifications (e.g., identification of the breathing pattern as irregular based on detection of irregularities in the breathing data (e.g., varying amplitudes of inhalation peaks)). In the example of FIG. 4, the breathing pattern data 126 can be further analyzed by breathing pattern analyzer 132 of FIGS. 1 and/or 3 with respect to, for example, generating user alert(s) 310.

FIG. 5 is a flowchart representative of example machine-readable instructions that, when executed, cause the example breathing pattern analyzer 132 of FIGS. 1 and/or 3 to analyze breathing pattern data generated from breathing sound data collected from a user (e.g., the user 104 of FIG. 1). The breathing pattern data can be generated by the example breathing pattern detector 122 of FIGS. 1 and/or 2 based on the instructions of FIG. 4. The example instructions of FIG. 5 can be executed by, for example, the second processor 128 of FIG. 1 to implement the breathing pattern analyzer 132 of FIGS. 1 and/or 3.

The rules manager 304 of the breathing pattern analyzer 132 of FIG. 3 analyzes the breathing pattern data 126 generated by the breathing pattern detector 122 based on the breath pattern rule(s) 306 (block 500). Based on the analysis, the rules manager 304 determines if alert(s) 310 should be generated (block 502). The rules manager 304 determines if thresholds and/or criteria for triggering the alert(s) 310 are satisfied. For example, the rules manager 304 can determine if a breathing rate satisfies a breathing rate threshold for providing an alert 310 the user. As another example, the rules manager 304 can determine whether the breathing data indicates a potential health condition such as an asthma attack that warrants an alert 310 to be delivered to the user. In other examples, the rules manager 304 determines that the breathing pattern data 126 should be always be provided to the user (e.g., when the user is wearing the HMD 102).

If the rules manager 304 determines that the alert(s) 310 should be generated, the alert generator 308 generates the alert(s) 310 for presentation via the HMD 102, a device carried by the HMD 102, and/or the user device 130 (block 504). The communicator 302 transmits the alert(s) 310 for presentation by the HMD 102, a device carried by the HMD 102, and/or the user device 130 in visual, audio, and/or tactile formats.

The example rules manager 304 continues to analyze the breathing pattern data 126 with respect to determining whether the alert(s) 310 should be generated (block 506). If there is no further breathing pattern data, the breathing pattern identifier 218 determine whether further breathing sound data 114 has been received at the breathing pattern detector 122 (block 508). In some examples, the collection of the breathing sound data 114 is controlled by the microphone manager 312 based on the microphone rule(s) 314 with respect to, for example, a duration for which the first microphone 106 collects the breathing sound data 114. If there is further breathing sound data, the breathing pattern detector 122 of FIGS. 1 and/or 2 modifies the breathing sound data to substantially remove noise and analyzes the breathing sound data as disclosed above in connection with FIG. 4. If there is no further breathing pattern data 126 and no further breathing sound data 114, the instructions of FIG. 4 end (block 510)

FIG. 6 is a block diagram of an example processor platform 600 capable of executing one or more of the instructions of FIG. 4 to implement the breathing pattern detector 122 of FIGS. 1 and/or 2. The processor platform 600 can be, for example, a server, a personal computer, a mobile device (e.g., a cell phone, a smart phone, a tablet such as an iPad™), a personal digital assistant (PDA), an Internet appliance, a wearable device such as eyeglasses including one or more processors coupled thereto, or any other type of computing device.

The processor platform 600 of the illustrated example includes a processor 122. The processor 122 of the illustrated example is hardware. For example, the processor 122 can be implemented by one or more integrated circuits, logic circuits, microprocessors or controllers from any desired family or manufacturer. The hardware processor may be a semiconductor based (e.g., silicon based) device. In this example, the processor 122 implements the example A/D converter 202, the example signal modifier 204, the example filter 210, the example signal adjuster 214, and/or the example breathing pattern identifier 218 of the example breathing pattern detector 122.

The processor 122 of the illustrated example includes a local memory 613 (e.g., a cache). The processor 122 of the illustrated example is in communication with a main memory including a volatile memory 614 and a non-volatile memory 616 via a bus 618. The volatile memory 614 may be implemented by Synchronous Dynamic Random Access Memory (SDRAM), Dynamic Random Access Memory (DRAM), RAMBUS Dynamic Random Access Memory (RDRAM) and/or any other type of random access memory device. The non-volatile memory 616 may be implemented by flash memory and/or any other desired type of memory device. Access to the main memory 614, 616 is controlled by a memory controller. The database 200 of the breathing pattern detector may be implemented by the main memory 614, 616 and/or the local memory 613.

The processor platform 600 of the illustrated example also includes an interface circuit 620. The interface circuit 620 may be implemented by any type of interface standard, such as an Ethernet interface, a universal serial bus (USB), and/or a PCI express interface.

In the illustrated example, one or more input devices 622 are connected to the interface circuit 620. The input device(s) 622 permit(s) a user to enter data and/or commands into the processor 122. The input device(s) can be implemented by, for example, an audio sensor, a microphone, a camera (still or video), a keyboard, a button, a mouse, a touchscreen, a track-pad, a trackball, isopoint and/or a voice recognition system.

One or more output devices 624 are also connected to the interface circuit 620 of the illustrated example. The output devices 624 can be implemented, for example, by display devices (e.g., a light emitting diode (LED), an organic light emitting diode (OLED), a liquid crystal display, a cathode ray tube display (CRT), a touchscreen, a tactile output device, a printer and/or speakers). The interface circuit 620 of the illustrated example, thus, typically includes a graphics driver card, a graphics driver chip and/or a graphics driver processor.

The interface circuit 620 of the illustrated example also includes a communication device such as a transmitter, a receiver, a transceiver, a modem and/or network interface card to facilitate exchange of data with external machines (e.g., computing devices of any kind) via a network 626 (e.g., an Ethernet connection, a digital subscriber line (DSL), a telephone line, coaxial cable, a cellular telephone system, etc.). In this example, the interface circuit 620 implements the communicator 222.

The processor platform 600 of the illustrated example also includes one or more mass storage devices 628 for storing software and/or data. Examples of such mass storage devices 628 include floppy disk drives, hard drive disks, compact disk drives, Blu-ray disk drives, RAID systems, and digital versatile disk (DVD) drives.

The coded instructions 632 of FIG. 4 may be stored in the mass storage device 628, in the volatile memory 614, in the non-volatile memory 616, and/or on a removable tangible computer readable storage medium such as a CD or DVD.

FIG. 7 is a block diagram of an example processor platform 700 capable of executing one or more of the instructions of FIG. 5 to implement the breathing pattern analyzer 132 of FIGS. 1 and/or 3. The processor platform 700 can be, for example, a server, a personal computer, a mobile device (e.g., a cell phone, a smart phone, a tablet such as an iPad™), a personal digital assistant (PDA), an Internet appliance, a wearable device such as eyeglasses including one or more processors coupled thereto, or any other type of computing device.

The processor platform 700 of the illustrated example includes a processor 132. The processor 132 of the illustrated example is hardware. For example, the processor 132 can be implemented by one or more integrated circuits, logic circuits, microprocessors or controllers from any desired family or manufacturer. The hardware processor may be a semiconductor based (e.g., silicon based) device. In this example, the processor 132 implements the example rules manager 304, the example alert generator 308, and/or the example microphone manager 312 of the example breathing pattern analyzer 132.

The processor 132 of the illustrated example includes a local memory 713 (e.g., a cache). The processor 132 of the illustrated example is in communication with a main memory including a volatile memory 714 and a non-volatile memory 716 via a bus 718. The volatile memory 714 may be implemented by Synchronous Dynamic Random Access Memory (SDRAM), Dynamic Random Access Memory (DRAM), RAMBUS Dynamic Random Access Memory (RDRAM) and/or any other type of random access memory device. The non-volatile memory 716 may be implemented by flash memory and/or any other desired type of memory device. Access to the main memory 714, 716 is controlled by a memory controller. The database 300 of the breathing pattern analyzer may be implemented by the main memory 714, 716 and/or the local memory 713.

The processor platform 700 of the illustrated example also includes an interface circuit 720. The interface circuit 720 may be implemented by any type of interface standard, such as an Ethernet interface, a universal serial bus (USB), and/or a PCI express interface.

In the illustrated example, one or more input devices 722 are connected to the interface circuit 720. The input device(s) 722 permit(s) a user to enter data and/or commands into the processor 132. The input device(s) can be implemented by, for example, an audio sensor, a microphone, a camera (still or video), a keyboard, a button, a mouse, a touchscreen, a track-pad, a trackball, isopoint and/or a voice recognition system.

One or more output devices 724 are also connected to the interface circuit 720 of the illustrated example. The output devices 724 can be implemented, for example, by display devices (e.g., a light emitting diode (LED), an organic light emitting diode (OLED), a liquid crystal display, a cathode ray tube display (CRT), a touchscreen, a tactile output device, a printer and/or speakers). The interface circuit 720 of the illustrated example, thus, typically includes a graphics driver card, a graphics driver chip and/or a graphics driver processor. The alert(s) 310 of the alert generator 308 may be exported via the interface circuit 720.

The interface circuit 720 of the illustrated example also includes a communication device such as a transmitter, a receiver, a transceiver, a modem and/or network interface card to facilitate exchange of data with external machines (e.g., computing devices of any kind) via a network 726 (e.g., an Ethernet connection, a digital subscriber line (DSL), a telephone line, coaxial cable, a cellular telephone system, etc.). In this example, the communicator 302 is implemented by the interface circuit 720.

The processor platform 700 of the illustrated example also includes one or more mass storage devices 728 for storing software and/or data. Examples of such mass storage devices 728 include floppy disk drives, hard drive disks, compact disk drives, Blu-ray disk drives, RAID systems, and digital versatile disk (DVD) drives.

The coded instructions 732 of FIG. 5 may be stored in the mass storage device 728, in the volatile memory 714, in the non-volatile memory 716, and/or on a removable tangible computer readable storage medium such as a CD or DVD.

From the foregoing, it will be appreciated that methods, systems, and apparatus have been disclosed to detect breathing patterns based on breathing sound data collected from a user wearing a non-obtrusive wearable device, such as eyeglasses. Disclosed examples include a first microphone disposed proximate to, for example, the bridge of the user's nose when the user is wearing the wearable device. Disclosed examples include a second microphone to collect ambient noise data from the environment in which the user is located and/or other sounds generated by the user (e.g., the user's voice). Disclosed examples modify breathing sound data collected from the user by the first microphone to remove noise collected by the first microphone. In disclosed examples, the breathing sound data is modified by deducting the ambient noise data collected by the second microphone from the breathing sound data. Thus, disclosed examples eliminate or substantially eliminate noise from the breathing sound data to improve accuracy in detecting the breathing pattern(s).

Disclosed examples analyze the resulting breathing sound data to detect breathing patterns based on, for example, characteristics of the signal data and metrics derived therefrom (e.g., breathing rate). In some disclosed examples, the breathing pattern data is analyzed further to determine if notifications should be provided to the user to monitor breathing performance. Disclosed examples provide the breathing pattern data and/or analysis results for presentation via the wearable device and/or another user device (e.g., a smartphone).

The following is a non-exclusive list of examples disclosed herein. Other examples may be included above. In addition, any of the examples disclosed herein can be considered in whole or in part, and/or modified in other ways.

Example 1 includes a wearable device including a frame to be worn by a user in an environment; a first microphone carried by the frame, the first microphone to collect breathing sound data from the user; a second microphone carried by the frame, the second microphone to collect noise data from the environment; and at least one processor. The at least one processor is to modify the breathing sound data based on the environmental noise data to generate modified breathing sound data and identify a breathing pattern based on the modified breathing sound data.

Example 2 includes the wearable device as defined in claim 1, wherein the first microphone is disposed proximate to a nose of the user when the user wears the wearable device.

Example 3 includes the wearable device as defined in examples 1 or 2, wherein the second microphone is spaced part from the first microphone.

Example 4 includes the wearable device as defined in examples 1 or 2, wherein the at least one processor is to modify the breathing sound data by removing the noise data from the breathing sound data.

Example 5 includes the wearable device as defined in example 1, wherein the modified breathing data includes peaks associated with inhalation by the user and peaks associated with exhalation by the user, the at least one processor to identify the breathing pattern by calculating a breathing rate based on the inhalation peaks and the exhalation peaks.

Example 6 includes the wearable device as defined in examples 1 or 2, wherein the second microphone is to collect the noise data at substantially a same time as the first microphone is to collect the breathing sound data.

Example 7 includes the wearable device as defined in example 1, wherein the at least one processor includes a digital signal processor.

Example 8 includes the wearable device as defined in examples 1, 2, or 5, wherein the at least one processor includes a first processor and a second processor, the first processor to transmit the modified breathing sound data to the second processor.

Example 9 includes the wearable device as defined in example 1, wherein the at least one processor is to identify the breathing pattern based on one or more of a breathing rate, a duration of inhalation by the user, or a duration of exhalation by the user.

Example 10 includes the wearable device as defined in example 1, wherein the at least one processor is to filter the modified breathing data and identify the breathing pattern based on the filtered modified breathing data.

Example 11 includes the wearable device as defined in example 1, wherein the wearable device includes eyeglasses.

Example 12 includes an apparatus including a signal modifier to modify breathing sound data collected from a user by removing environmental noise data and generate modified breathing sound data. The example apparatus includes a breathing pattern identifier to identify a breathing pattern based on the modified breathing sound data to generate breathing pattern data and an alert generator to generate an alert based on the breathing pattern data.

Example 13 includes the apparatus as defined in example 12, further including a rules manager to analyze the breathing pattern data, the alert generator to generate the alert based on the analysis.

Example 14 includes the apparatus as defined in example 12, wherein the rules manager is to perform a comparison of the breathing pattern data to a threshold, the alert generator to generate the alert based on the comparison.

Example 15 includes the apparatus as defined in examples 12 or 13, further including a filter to filter the modified breathing sound data.

Example 16 includes the apparatus as defined in example 15, wherein the filter is a bandpass filter.

Example 17 includes the apparatus as defined in example 12 or 13, wherein the breathing pattern identifier is to identify the breathing pattern based on one or more of an amplitude of peaks or a frequency of peaks in the modified breathing data.

Example 18 includes the apparatus as defined in example 17, wherein the peaks include a first peak associated with inhalation and a second peak associated with exhalation.

Example 19 includes the apparatus as defined in example 12, wherein the breathing pattern identifier is to calculate a breathing rate based on the modified breathing data, the breathing pattern data to include the breathing rate.

Example 20 includes the apparatus of example 12, further including a communicator to transmit the breathing pattern data to a user device.

Example 21 includes the apparatus of example 12, further including a communicator to transmit the alert for presentation via a user device.

Example 22 includes at least one non-transitory computer readable storage medium including instructions that, when executed, cause a machine to at least modify breathing sound data collected from a user by removing environmental noise data; generate modified breathing sound data; identify a breathing pattern based on the modified breathing sound data to generate breathing pattern data; and generate an alert based on the breathing pattern data.

Example 23 includes the at least one non-transitory computer readable storage medium as defined in example 22, wherein the instructions cause the machine perform a comparison of the breathing pattern data to a threshold and generate the alert based on the comparison.

Example 24 includes the at least one non-transitory computer readable storage medium as defined in examples 22 or 23, wherein the instructions cause the machine to apply a bandpass filter to the modified breathing sound data.

Example 25 includes the at least one non-transitory computer readable storage medium as defined in examples 22 or 23, wherein the instructions cause the machine to identify the breathing pattern based on one or more of an amplitude of peaks or a frequency of peaks in the modified breathing data.

Example 26 includes the at least one non-transitory computer readable storage medium as defined in example 25, wherein the peaks include a first peak associated with inhalation and a second peak associated with exhalation.

Example 27 includes the at least one non-transitory computer readable storage medium as defined in example 22, wherein the instructions cause the machine to calculate a breathing rate based on the modified breathing data, the breathing pattern data to include the breathing rate.

Example 28 includes the at least one non-transitory computer readable storage medium as defined in example 22, wherein the instructions cause the machine to transmit the breathing pattern data to a user device.

Example 29 includes the at least one non-transitory computer readable storage medium as defined in example 22, wherein the instructions cause the machine to transmit the alert for presentation via a user device.

Example 30 includes a method including modifying breathing sound data collected from a user by removing environmental noise data, generating modified breathing sound data, identifying a breathing pattern based on the modified breathing sound data to generate breathing pattern data, and generating an alert based on the breathing pattern data.

Example 31 includes the method as defined in example 30, further including performing a comparison of the breathing pattern data to a threshold and generating the alert based on the comparison.

Example 32 includes the method as defined in examples 30 or 31, further including applying a bandpass filter to the modified breathing sound data.

Example 33 includes the method as defined in examples 30 or 31, further including identifying the breathing pattern based on one or more of an amplitude of peaks or a frequency of peaks in the modified breathing data.

Example 34 includes the method as defined in example 33, wherein the peaks include a first peak associated with inhalation and a second peak associated with exhalation.

Example 35 includes the method as defined in example 30, further including calculating a breathing rate based on the modified breathing data, the breathing pattern data to include the breathing rate.

Example 36 includes the method as defined in example 30, further including transmitting the breathing pattern data to a user device.

Example 37 includes the method as defined in example 30, further including transmitting the alert for presentation via a user device.

Example 38 includes an apparatus including means for modifying breathing sound data obtained from a user by removing environmental sound data to generate modified breathing sound data; means for identifying a breathing pattern based on the modified breathing sound data; and means for generating an alert based on the modified sound data.

Example 39 includes the apparatus as defined in example 38, wherein the means for modifying the breathing sound data includes a digital signal processor.

Example 40 includes the apparatus as defined in example 39, wherein the digital signal processor is carried by a wearable device.

Example 41 includes the apparatus as defined in example 38, further including means for transmitting the alert to a user device.

Example 42 includes the apparatus as defined in example 38, further including means for bandpass filtering the modified breathing data.

Example 43 includes an apparatus including means for obtaining breathing sound data from a user; means for obtaining environmental data from an environment in which the user is located; means for modifying the breathing sound data based on the environmental data to generate modified breathing sound data; and means for identifying a breathing pattern based on the modified breathing sound data.

Example 44 includes the apparatus as defined in example 43, wherein the means for obtaining the breathing sound data is a first microphone coupled to a wearable device and the means for obtaining the environmental data is a second microphone coupled to the wearable device.

Example 45 includes the apparatus as defined in example 44, wherein the wearable device includes eyeglasses.

Example 46 includes the apparatus as defined in example 44, further including means for controlling a duration of time that the first microphone is to collect the breathing sound data.

Example 47 includes the apparatus as defined in example 43, wherein the means for modifying the breathing sound data is to deduct the environmental noise data from the breathing sound data to generate the modified breathing sound data.

Although certain example methods, apparatus and articles of manufacture have been disclosed herein, the scope of coverage of this patent is not limited thereto. On the contrary, this patent covers all methods, apparatus and articles of manufacture fairly falling within the scope of the claims of this patent.

What is claimed is:

1. A wearable device comprising:
a frame to be worn by a user in an environment;
a first microphone carried by the frame, the first microphone to collect breathing sound data from the user;
a second microphone carried by the frame, the second microphone to collect noise data from the environment; and
at least one processor to:
modify the breathing sound data based on the environmental noise data to generate modified breathing sound data; and
identify a breathing pattern based on the modified breathing sound data.

2. The wearable device as defined in claim 1, wherein the first microphone is disposed proximate to a nose of the user when the user wears the wearable device.

3. The wearable device as defined in claim 1, wherein the at least one processor is to modify the breathing sound data by removing the noise data from the breathing sound data.

4. The wearable device as defined in claim 1, wherein the second microphone is to collect the noise data at substantially a same time as the first microphone is to collect the breathing sound data.

5. The wearable device as defined in claim 1, wherein the at least one processor includes a first processor and a second processor, the first processor to transmit the modified breathing sound data to the second processor.

6. The wearable device as defined in claim 1, wherein the at least one processor is to identify the breathing pattern based on one or more of a breathing rate, a duration of inhalation by the user, or a duration of exhalation by the user.

7. The wearable device as defined in claim 1, wherein the wearable device includes eyeglasses.

8. An apparatus comprising:
a signal modifier to:
modify breathing sound data collected from a user by removing environmental noise data; and
generate modified breathing sound data;
a breathing pattern identifier to identify a breathing pattern based on the modified breathing sound data to generate breathing pattern data; and
an alert generator to generate an alert based on the breathing pattern data.

9. The apparatus as defined in claim 8, further including a rules manager to analyze the breathing pattern data, the alert generator to generate the alert based on the analysis.

10. The apparatus as defined in claim 8, wherein the rules manager is to perform a comparison of the breathing pattern data to a threshold, the alert generator to generate the alert based on the comparison.

11. The apparatus as defined in claim 8, further including a filter to filter the modified breathing sound data.

12. The apparatus as defined in claim 8, wherein the breathing pattern identifier is to identify the breathing pattern based on one or more of an amplitude of peaks or a frequency of peaks in the modified breathing data.

13. The apparatus as defined in claim 8, wherein the breathing pattern identifier is to calculate a breathing rate based on the modified breathing data, the breathing pattern data to include the breathing rate.

14. At least one non-transitory computer readable storage medium comprising instructions that, when executed, cause a machine to at least:
modify breathing sound data collected from a user by removing environmental noise data;
generate modified breathing sound data;
identify a breathing pattern based on the modified breathing sound data to generate breathing pattern data; and
generate an alert based on the breathing pattern data.

15. The at least one non-transitory computer readable storage medium as defined in claim 14, wherein the instructions cause the machine perform a comparison of the breathing pattern data to a threshold and generate the alert based on the comparison.

16. The at least one non-transitory computer readable storage medium as defined in claim 14, wherein the instructions cause the machine to apply a bandpass filter to the modified breathing sound data.

17. The at least one non-transitory computer readable storage medium as defined in claim 14, wherein the instructions cause the machine to identify the breathing pattern based on one or more of an amplitude of peaks or a frequency of peaks in the modified breathing data.

18. The at least one non-transitory computer readable storage medium as defined in claim 14, wherein the instructions cause the machine to calculate a breathing rate based on the modified breathing data, the breathing pattern data to include the breathing rate.

19. The at least one non-transitory computer readable storage medium as defined in claim 14, wherein the instructions cause the machine to transmit the breathing pattern data to a user device.

20. The at least one non-transitory computer readable storage medium as defined in claim 14, wherein the instructions cause the machine to transmit the alert for presentation via a user device.
